(19) United States
(12) Patent Application Publication
(10) Pub. No.: US 2011/0123627 A1
(43) Pub. Date: May 26, 2011

(54) HIGH DENSITY COMPOSITIONS CONTAINING POSACONAZOLE AND FORMULATIONS COMPRISING THE SAME

(76) Inventors: Larry Yun Fang, Plainsboro, NJ (US); David Harris, New Providence, NJ (US); Gopal Krishna, North Brunswick, NJ (US); Allen E. Moton, JR., East Hanover, NJ (US); Russell C. Prestipino, Hoboken, NJ (US); Marc Steinman, Livingston, NJ (US); Jiansheng Wan, Warren, NJ (US); Hetty Anne Waskin, Skillman, NJ (US)

(21) Appl. No.: 12/999,547

(22) PCT Filed: Apr. 15, 2009
(86) PCT No.: PCT/US09/40652

§ 371 Date: Dec. 16, 2010

Related U.S. Application Data
(60) Provisional application No. 61/045,177, filed on Apr. 15, 2008.
(60) Provisional application No. 61/166,487, filed on Apr. 3, 2009.

(51) Int. Cl. A61K 31/496; A61K 9/14; A61P 31/10

(52) U.S. Cl. 424/489; 514/254.07

(57) ABSTRACT

The present application provides novel compositions comprising posaconazole and a polymer wherein the composition has a glass transition temperature (Tg) of less than about 1100 C. The application also describes compositions comprising posaconazole and a polymer having a bulk density of greater than about 0.4 mg/mL. The application also describes compositions comprising posaconazole and a polymer which provide an exposure (AUCtf) of at least about 10,000 ng·hr/mL when administered to a patient in a fasted state. The application also describes a novel process for preparing these compositions. The preff erred polymer is HPMCAS. Preferably the composition is an extruded material.

### FIELD OF THE INVENTION

This application discloses novel solid composition comprising posaconazole and pharmaceutical formulations comprising the same.

### BACKGROUND OF THE INVENTION

Identification of any publication in this section or any section of this application is not an admission that such publication is prior art to the present invention.

Posaconazole is an azole compound with antifungal properties. The compound and its synthesis are described in, for example, U.S. Pat. No. 5,703,079 (issued Dec. 30, 1997) and related U.S. Pat. No. 5,661,151 (issued Aug. 26, 1997) both to Saksena et al. A stable crystalline form of posaconazole and a process for preparing the crystalline form are describe in U.S. Pat. No. 6,958,337, issued Oct. 25, 2005 to Andrews et al. A pharmaceutical formulation comprising a suspension of this crystalline form (available commercially as Noxafil®) and a method for preparing the same are described in published U.S. Patent Application No. 2003/0055067, filed Apr. 1, 2002 and published Mar. 20, 2003.

A suspension containing posaconazole in crystalline form (40 mg/mL) has been approved inter alia in the U.S. and European Union as Noxafil® for oral administration in the treatment of invasive fungal infections, for example, the treatment of oropharangyl candidiasis, including infections which are resistant to treatment by other azole antifungals, and as a prophylatic treatment to prevent fungal infections in patients who are who are at high risk of developing these infections due to being severely immunocompromised, such as hematopoietic stem cell transplant (HSCT) recipients with graft-versus-host disease (GVHD) or those with hematologic malignancies with prolonged neutropenia from chemotherapy. Noxafil® is indicated for oral administration with food, preferably a high-fat meal (or in severely neutropenic patients unable to tolerate food intake, after administration of a nutritional supplement) in order to assure attainment of adequate plasma concentrations of posaconazole. As reported in the PDR, administration of Noxafil® to a patient with a high-fat meal exhibits an increase in drug plasma concentration of 4× over what is observed after administration of an equivalent amount of Noxafil® to a fasting patient (also termed herein, “Fasted Conditions”), and exhibits increases of 3× in plasma concentration when administered to patients along with a nutritional supplement over what is observed after administration of Noxafil® to a fasted patient. Administration of posaconazole formulations accompanied by either a meal or a nutritional supplement are collectively referred to herein also as administration under “Fed Conditions”.

The provision of a solid composition comprising posaconazole suitable for preparing a solid dosage form for oral administration has heretofore been hampered by the poor solubility and weak basicity of the posaconazole free-base compound. Posaconazole is soluble at low pH. For example, in the environment of the stomach (approximately pH 1) posaconazole free base has a solubility of approximately 0.8 mg/mL. However, when posaconazole dissolved in the stomach fluids reaches the environment of the intestines (typically less acidic than about pH 6.4) a substantial amount of the dissolved posaconazole precipitates, hindering absorption in the intestines. It has been determined that in environments where the pH is about pH 6.4 or more basic the solubility of posaconazole free base is less than about 1 microgram/mL.

Hydroxypropylmethylcellulose-derivative polymers (HPMC-derivative polymers) have been examined as a means of providing a composition imparting improved bioavailability when used in formulations where the active pharmaceutical ingredient (API) is absorbed in the intestine but is poorly or sparingly soluble in the intestinal environment. U.S. Pat. No. 7,235,260, issued Jun. 26, 2007 to Crew et al. (the '260 patent), describes glycogen phosphorlase inhibitors in hydroxypropylmethylcellulose and hydroxypropylmethylcellulose-derivative polymers. The compositions described in the '260 patent are prepared by spray-drying a solution containing a phosphorlase inhibitor and hydroxypropylmethylcellulose acetate succinate (HPMC-AS) dissolved in a common solvent. U.S. Pat. No. 6,881,745 (the '745 patent) issued Apr. 19, 2005 to Hayes et al., generally describes compositions comprising an azole antifungal compound and a polymer. The compositions described are prepared by dissolving the azole compound and polymer in a common solvent, for example, methylene chloride, chloroform, ethanol, methanol, isopropanol, ethylacetate, or acetone, or mixtures of two or more thereof, and forming a solid granular composition by spray-drying the solution using conventional spray-drying equipment. An example of an azole-containing composition described in the '745 patent is itraconazole with a hydroxypropylmethylcellulose-phthalate (HPMC-phthalate) polymer derivative prepared by spray-drying a solution containing the active pharmaceutical ingredient (API) and the polymer. These compositions are reported to show improvement in itraconazole bioavailability and elimination of a food effect connected with the administration of itraconazole.

### SUMMARY OF THE INVENTION

It is desirable to have a formulation for oral administration which provides posaconazole to a patient population with lower patient to patient variability in bioavailability, thus providing consistent PK parameters across a patient population to whom the formulation is administered, for example a narrower observed range for Cₘₐₓ and AUC values across a population of patients to whom a fixed amount of a formulation is administered. Moreover, it is desirable to have a formulation for oral administration which provides higher posaconazole bioavailability than is available from prior formulations, thus yielding higher plasma levels determined from blood obtained from a patient to whom a given amount of posaconazole is administered (termed herein also for convenience “plasma level(s)”). In addition, it is desirable to have a formulation for oral administration which provides an acceptable plasma level of posaconazole when administered to a patient in a fasted state.

What is needed is a posaconazole composition and a pharmaceutical formulation comprising a posaconazole composition that is suitable for oral administration to patients under fasted conditions, and which provides therapeutic plasma level(s) and sufficient posaconazole exposure (AUC) to yield a therapeutic benefit. Additionally, what is needed is a pharmaceutical formulation for oral administration which provides posaconazole in a form that is essentially insoluble when passing through the stomach environment, but which readily releases posaconazole once it has passed into the environment of the small intestine. What is needed also is a formulation that upon administration to a cross-section of patients, exhibits less patient to patient variability in pharmacokinetic parameters (PK) than is available from prior formulations.

These needs and other objectives and/or advantages are provided by the present invention, which in one aspect provides a novel composition comprising posaconazole dissolved or molecularly dispersed in a hydroxypropylmethylcellulose-derivative polymer (HPMC-derivative polymer). In some embodiments it is preferred for the HPMC-derivative polymer to be hydroxypropylmethylcellulose acetate succinate polymer (HPMC-AS). In some embodiments it is preferred to provide a composition which has a solid density of at least about 1.2 g/mL. In some embodiments it is preferred to provide a particulate form of the composition, wherein the particulate form has a bulk density of at least about 0.6 g/ml and provides an AUC(tf) of at least about 10,000 hr·ng/mL, or a Cₘₐₓ of at least about 300 ng/mL when an amount of the composition comprising an amount of posaconazole equivalent to about 100 mg of posaconazole free-base is administered to a patient in a fasted state. In some embodiments it is preferred to provide a milled composition of the invention having a bulk density of from about 0.6 g/mL to about 0.7 g/mL. In some embodiments it is preferred to provide a particulate form of the composition which, when administered under fasted conditions to a patient in an amount comprising from about 80 to about 500 mg of posaconazole, preferably from about 100 mg to about 400 mg of posaconazole, yields a Cₘₐₓ of at least about 300 ng/mL, preferably at least about 335 ng/mL. In some embodiments it is preferred to provide a dosage form having from about 80% to about 125% of the desired amount of posaconazole, in accordance with US FDA standards for manufacturing medicaments.

As the term is used herein “bulk density” has its conventional meaning, and preferably bulk density is determined by weighing a measured volume of the material in particle form (herein termed also “the volumetric method”). As the term is used herein, “solid density” refers to the weight/solid volume occupied by a sample of the material. One method of determining solid density is to place a weighed sample of the material into a liquid having a lower density than the solid and in which the solid is insoluble, thereby permitting the solid volume of the material to be determined by the amount of liquid displaced by the solid, and dividing the weight of the sample by the measured volume of the sample. It will be appreciated that other methods for determining solid density and the bulk density yielding at least comparable accuracy may be used.

In some embodiments it is preferred to select an HPMC-derivative polymer in which the free base form of posaconazole is soluble and wherein the polymer has a glass transition temperature (T_{g}) of from about 120° C. to about 137° C. In some embodiments it is preferred to select a polymer in which the free base form of posaconazole is soluble and wherein the polymer has a T_{g} of from about 120° C. to about 135° C. In some embodiments it is preferred to select hydroxypropyl methyl cellulose-acetate succinate (HPMC-AS) as the HPMC-derivative polymer, preferably an HPMC-AS polymer that has a glass transition temperature of from about 120° C. to about 130° C. In some embodiments it is preferred to select a polymer in which posaconazole is soluble and which upon dissolution of posaconazole into the polymer behaves as a eutectic mixture having a melting point below the melting point of posaconazole.

In some embodiments employing HPMC-AS, it is preferred to select a polymer having a degree of polymerization of about 70. In some embodiments it is preferred to select as the HPMC-AS polymer at least one of: (i) an HPMC-AS polymer having an average of 8 wt % acetyl content and 15 wt. % succinoyl content; (ii) an HPMC-AS polymer having an average of 9 wt. % acetyl content and 11 wt. % succinoyl content; or (iii) an HPMC-AS polymer having an average of 12 wt. % acetyl content and 6 wt. % succinoyl content, more preferably an HPMC-AS polymer having a degree of polymerization of about 70 and an average of 9 wt. % acetyl content and 11 wt. % succinoyl content.

In some embodiments it is preferred to employ a type of an HPMC-AS polymer and in an amount that yields an HPMC-AS/posaconazole composition of the invention which has a glass-transition temperature (T_{g}) of less than about 110° C., preferably a T_{g} of from about 70° C. to about 110° C., more preferably a T_{g} of from about 80° C. to about 95° C. In some embodiments employing HPMC-AS it is preferred to include in the composition an amount of posaconazole free base yielding an HPMC-AS:Posaconazole free base ratio equal to from about 95 wt. % HPMC-AS:5 wt. % posaconazole free base, to about 50 wt. % HPMC-AS:50 wt. % posaconazole free base. In some embodiments it is preferred to have the wt. ratio of posaconazole free base and HPMC-AS of about 1:3, posaconazole:HPMC-AS.

In some embodiments the compositions of the invention comprise additionally: one or more plasticizers, for example vitamin E, steric acid, or TEC (triethyl citrate); one or more preservatives and/or antioxidants, for example, vitamin C or/and butylated hydroxytoluene (BHT).

Another aspect of the present invention is a process for preparing a composition comprising posaconazole free base molecularly dispersed in or dissolved in a hydroxypropylmethylcellulose-derivative polymer. In some embodiments it is preferred to select the polymer used in a composition of the invention from those HPMC-derivative polymers providing the following properties: (i) posaconazole is soluble in the polymer; (ii) posaconazole forms a solution or dispersion behaving as a eutectic which has a melting point below the melting point of posaconazole; (iii) when posaconazole is admixed with the selected polymer(s) and heated it apparently acts as a fluxing agent to promote melting the polymer and promote dissolution of posaconazole into the polymer. In some embodiments the process for preparing a composition of the invention comprises: (i) forming a admixture of posaconazole and the selected polymer; (ii) forming a molten dispersion by heating the admixture to a temperature above about 60° C. and below about 169° C., optionally with stirring of the molten dispersion; (iii) cooling the dispersion provided in Step (ii) to form a solid; and (iv) optionally forming a shaped mass from the dispersion either before or contemporaneously with Cooling Step (iii). In some embodiments it is preferred for the HPMC-derivative polymer to be a hydroxypropylmethylcellulose acetate succinate (HPMC-AS) polymer. In some embodiments in which the polymer selected is HPMC-AS it is preferred to prepare the composition by a process comprising:
(i) dry-blending a mixture of granules or particles of posaconazole free-base and the hydroxypropylmethylcellulose acetate succinate polymer (HPMC-AS), thereby forming an admixture;
(ii) forming a molten dispersion of the posaconazole free base dissolved in HPMC-AS polymer, by heating the admixture from Step (i) to a temperature which is: above the glass transition temperature (T_{g}) of posaconazole (preferably above about 60° C.), preferably above the T_{g} of a molecular dispersion comprising posaconazole in HPMC-AS where the ratio of posaconazole and HPMC-AS in the dispersion is equal to the ratio of posaconazole and HPMC-AS in the admixture provided in Step (i), more preferably above the T_{g} of the HPMC-AS used to prepare the admixture in step (i); and a temperature which is below the melting point of posaconazole free base (generally about 169° C.), preferably the molten dispersion is formed at a temperature of from about 80° C. to about 160° C., more preferably at a temperature of from about 120° C. to about 160° C., and optionally blending the admixture while heating;
(iii) cooling the molten dispersion formed in Step (ii) to provide a solid composition of posaconazole free base molecularly dispersed or dissolved in an HPMC-AS polymer;
(iv) optionally, prior to, or during cooling Step (iii), forming the dispersion prepared in Step (ii) into a shaped mass, preferably forming it into an extruded shape; and
(v) optionally milling or optionally granulating the solid composition provided in Step (iii), or if optional step (iv) has been carried out, optionally milling or optionally granulating the extruded shapes provided in Step (iv), to form a particulate product.

In some embodiments it is preferred to prepare compositions of posaconazole free base/HPMC-AS polymer selected to release less than about 10 mole % of the dissolved posaconazole within one hour when an aliquot of the composition is maintained in an environment equal to pH 1 and releases more than about 20 mole % of the dissolved or dispersed posaconazole present in the aliquot when maintained in an environment equal to a pH of from about pH 6.0 to about pH 7.0. This dissolution behaviour is illustrated in FIG. 1A for a pH 1 environment and in FIG. 1B for a pH 6.4 environment. In some embodiments it is preferred to measure the posaconazole dissolution profile by placing an aliquot of the composition into a dissolution medium comprising an aqueous HCl solution contained in a paddle dissolution apparatus which has a pH of about pH 1.0, and stir the mixture for a first period of stirring lasting about 60 minutes at a paddle speed of 100 RPM while extracting aliquots of the dissolution medium and analyzing them for dissolved posaconazole. In determinations carried out using this method, is it preferred to raise the pH of the dissolution medium at the end of the first period of stirring by adding a mixture of monobasic sodium phosphate and dibasic sodium phosphate salts (Na₂HPO₄ and NaH₂PO₄) in sufficient quantity to produce a dissolution medium having a pH of from about 6.4 to about 6.8 and continue the stirring while extracting and analyzing aliquots of the dissolution solvent for dissolved posaconazole. In some embodiments it is preferred to carry out dissolution tests using a USP dissolution apparatus II (paddle dissolution apparatus) in conjunction with the above-described procedure.

In another aspect the present invention provides a dosage form comprising a composition comprising posaconazole free base dissolved in, or molecularly dispersed in, an HPMC-derivative polymer. In some embodiments it is preferred to directly incorporate the composition as prepared into a dosage form, for example, placing an extruded shape or a particulate from of a composition of the invention into a capsule without any additional excipients. In some embodiments of the process in which an extrusion step is included in the process, it is preferred to directly extrude the molten dispersion into a capsule without additional excipients to provide a dosage form comprising the composition of the invention. In some embodiments it is preferred to mill a solid form of the composition, for example, milling an extrudate form of the composition, to provide a particulate form of the composition. In some embodiments it is preferred to provide the composition in a granular form. In some embodiments it is preferred to mix a milled particulate form or a granular form of the composition of the invention with one or more excipients and press the mixture into a tablet dosage form or charge the mixture into capsules. In some embodiments it is preferred to form a composition comprising posaconazole free base dissolved in, or molecularly dispersed in HPMC-AS in particulate form and directly place a quantity of the particulate material in a capsule without additional excipients.

The invention also provides methods of prophylactically or therapeutically treating fungal infections by administering a quantity of: a composition of the invention; formulation comprising a composition of the invention; or dosage form comprising a composition of the invention, which administered quantity provides from about 80 mg to about 500 mg of posaconazole per day, either in a single or divided dose. In some embodiments it is preferred to administer daily, in either a single or divided dose an amount of: a composition of the invention; formulation comprising a composition of the invention; or dosage form comprising a composition of the invention which provides from about 100 mg to about 400 mg of posaconazole, preferably at least about 200 mg of posaconazole. In some embodiments wherein it is preferred to provide treatment by administering from about 100 mg of posaconazole to about 400 mg of posaconazole per day, it is preferred to supply a medicament comprising an amount of a composition of the invention providing from about 80% to about 125% of the amount of posaconazole desired for providing treatment.

In some embodiments it is preferred to administer a composition of the invention in an amount and over an interval which provides a steady-state average plasma concentration (C_{avg}) of at least about 319 ng/ml in at least about 75% of a patient population to whom it is administered. In some embodiments it is preferred to administered a composition of the invention, a formulation comprising a composition of the invention, or a dosage form comprising a composition of the invention in an amount providing from about 100 mg of posaconazole to about 400 mg of posaconazole daily, in a single or divided dose, preferably in a divided dose, BID, for a period of from about 5 to about 10 days to achieve a steady-state average plasma concentration (C_{avg}) of at least about 319 ng/mL in at least about 75% of a patient population to whom it is administered, or a steady-state average plasma concentration (C_{avg}) of at least about 228 ng/mL in at least about 90% of a patient population to whom it is administered.

Other aspects and advantages of the invention will become apparent from following Detailed Description and the appended figures.

### BRIEF DESCRIPTION OF THE FIGURES

The present invention is more fully described in the following detailed description and the appended figures in which:

FIG. 1A presents a graphic representation of a dissolution profile at pH 1 (0.1 N HCl) for dosage forms prepared using three different compositions of the invention as determined using a USP Apparatus II with a 100 RPM paddle speed.

FIG. 1B presents a graphic representation of a dissolution profile at pH 6.4 (50 mM phosphate buffer) for a dosage form prepared using three different compositions of the invention as determined using a USP Apparatus II with a 100 RPM paddle speed.

FIG. 2 presents a graphic representation of Differential Scanning Calorimetry (DSC) data obtained from a composition of the invention comprising a 1:3 wt. ratio of posaconazole:HPMC-AS (M-grade).

FIG. 3 presents a graphic representation of an X-Ray Powder Diffraction Spectrum of a composition of the invention comprising different wt. ratios (1:1, 1:2, 1:3, and 1:4) of posaconazole:HPMC-AS (M-grade) plotted as intensity versus diffraction angle (expressed as 2°-theta).

FIG. 4A presents a graphic representation of the plasma levels observed after administering to human subjects a 100 mg dose under “Fed Conditions” of: (i) a posaconazole suspension (Noxafil®); (ii) a milled granular posaconazole/HPMC-AS polymer composition of the invention contained in a capsule; and (iii) a milled granular posaconazole/HPMC-AS polymer composition of the invention incorporated into two different formulations and pressed into tablets.

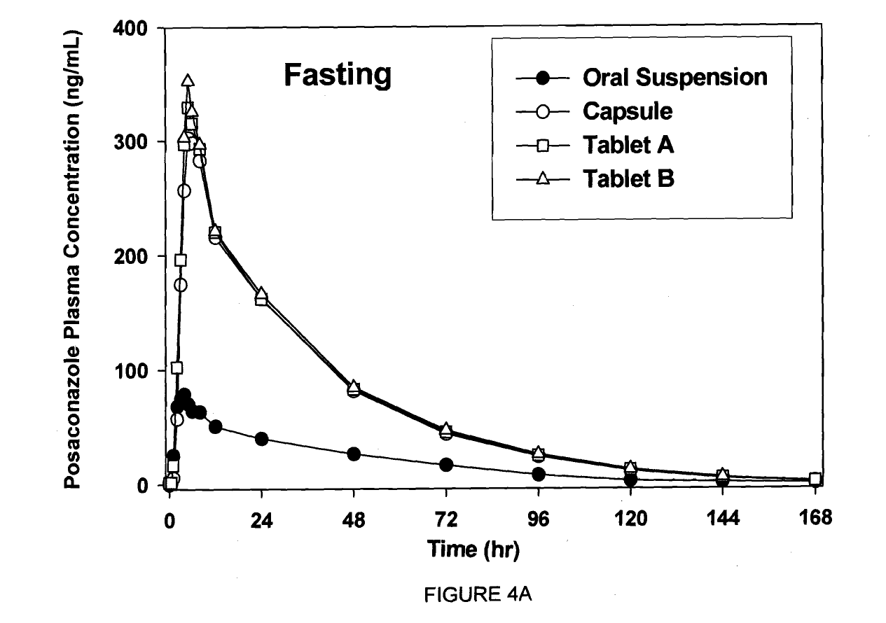

FIG. 4B presents a graphic representation of the plasma levels observed after administering to human subjects a 100 mg dose under “Fasted Conditions” of: (i) a posaconazole suspension (Noxafil®); (ii) a milled granular posaconazole/HPMC-AS polymer composition of the invention contained in a capsule; and (iii) a milled granular posaconazole/HPMC-AS polymer composition of the invention incorporated into two different formulations and pressed into tablets.

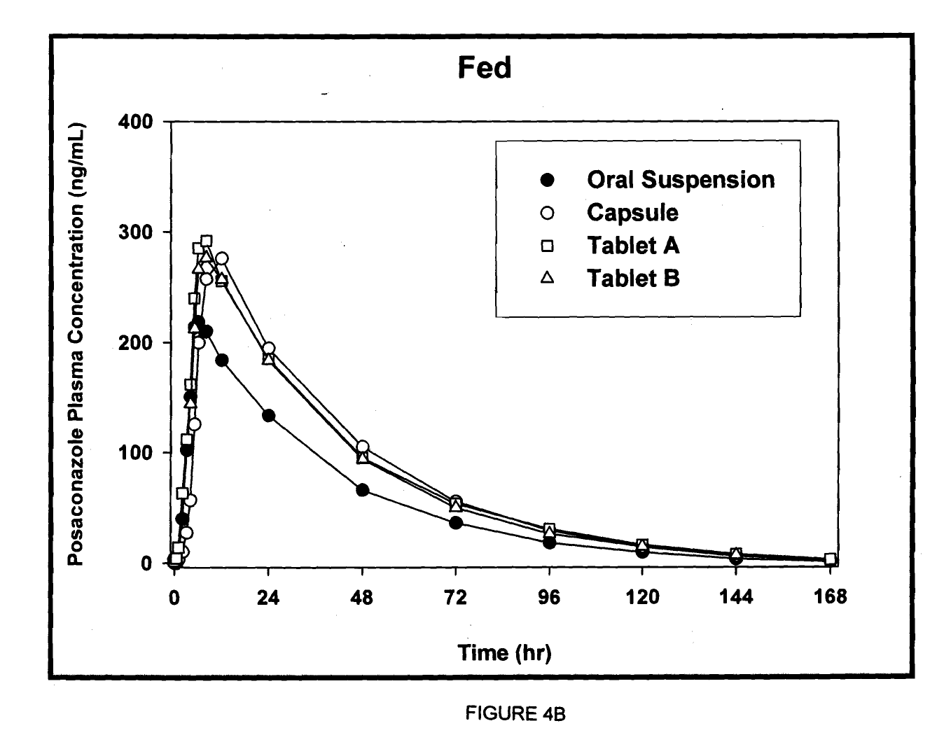

FIG. 5 presents a graphic representation of the increasing rate of posaconazole degradation when it is present in polymer melts of increasing temperature for a fixed amount of time at temperature (% increase in HPLC signal of degradant).

### DETAILED DESCRIPTION OF THE INVENTION

As mentioned above, in one aspect the present invention is a composition comprising posaconazole and a polymer, wherein the composition has a solid density of greater than about 1.2 g/mL. Without wanting to be bound by theory, it is believed that in the compositions of the invention the posaconazole is present either dissolved in or molecularly dispersed in the polymer. Without wanting to be bound by theory it is believed that the inventive compositions display either solid solution morphology, albeit it with a very low degree of long-range ordering, or the compositions of the invention are essentially amorphous and therefore have the morphology of a glass material. Any and all of these morphologies are contemplated herein by the terms “dissolved in”, “molecularly dispersed in”, “molecular dispersion”, “molten dispersion”, and “dispersion”, used herein for convenience to describe the compositions of the invention in various stages of preparation and at various temperatures.

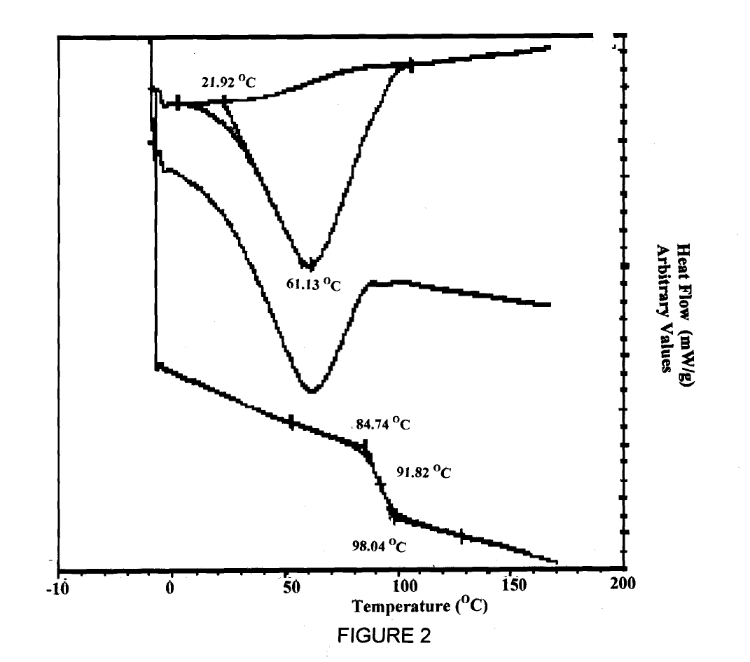

The morphology of a composition of the invention is better understood in part with reference to FIG. 2, which presents the results of differential scanning calorimetry (DSC) obtained using a sample of a composition of the invention having a weight ratio of HPMC-AS polymer:posaconazole free base of 3:1. FIG. 2 shows that these samples display a single endotherm centered at about 90° C., which is consistent with the melting point (mp) or glass transition temperature (T_{g}) of a material having a single phase, for example, a solid solution or a glass material. The inventors have found similar DSC behavior using sample of compositions of the invention which contain a weight ratio of HPMC-AS polymer:posaconazole free base of from about 4:1 polymer:posaconazole to about 1:1 polymer:posaconazole.

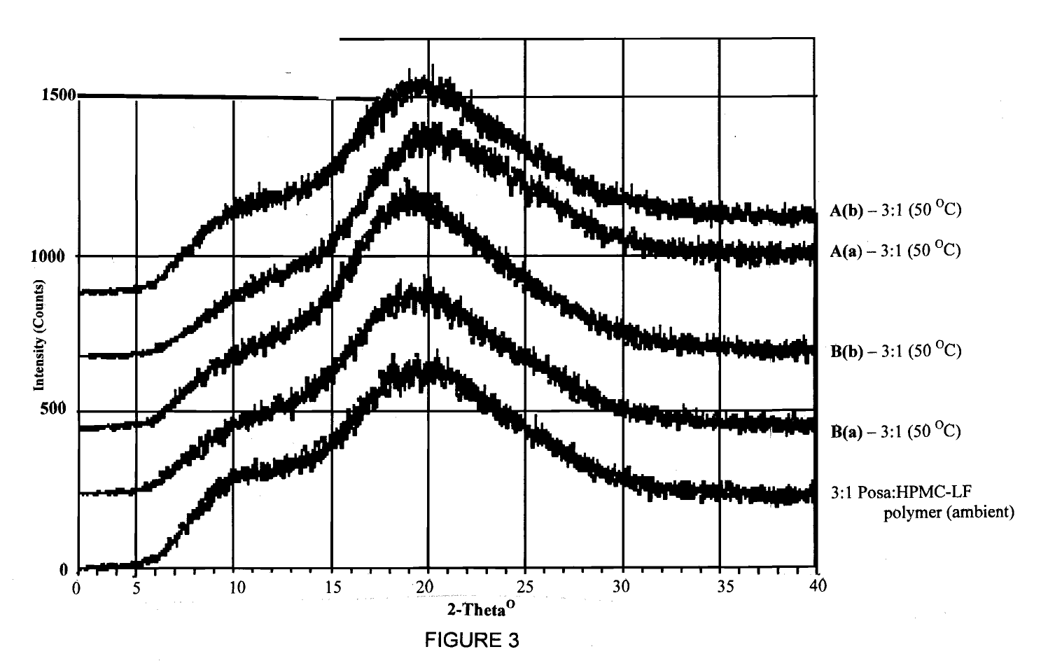

The absence of crystallinity in a composition of the invention can be better understood with reference to FIG. 3, which presents a pair of XRD powder patterns obtained from each of two different compositions of the invention. Accordingly, spectra A(a) and A(b) shown in FIG. 3 present XRD data obtained using a composition of the invention comprising a weight ratio of HPMC-AS polymer:posaconazole free base of 3:1. Spectrum A(a) presents the data obtained using a sample of the composition aged for three months at room temperature and spectrum A(b) presents the data obtained using a sample of the composition aged for three months at a 50° C. storage temperature. Spectra B(a) and B(b) shown in FIG. 3 present XRD data obtained using a composition of the invention comprising a weight ratio of HPMC-AS polymer:posaconazole free base of 1:1. Spectrum B(a) presents data obtained from a sample of the composition aged at room temperature for 3 months and spectrum B(b) presents data obtained using a sample of the composition aged for three months at a 50° C. storage temperature. FIG. 3 also contains, as the lowest trace on the figure, a diffraction pattern obtained from a sample of a composition of the invention made with a different grade of HPMC-AS polymer (grade L) which is present at a 3:1 ratio with respect to posaconazole.

The spectra of FIG. 3 indicate that no crystalline posaconazole was detected in any of the samples even after storage under heated conditions. The XRD technique employed has a limit of detection of crystalline phases of about 3 wt. % of sample, accordingly, these data indicate that if compositions of the invention comprise any crystalline posaconazole, the amount present is less than about 3 wt. % of the sample examined. The XRD data of FIG. 3 and DSC data of FIG. 2 taken together indicate that a composition of the invention has a single phase and little or no long range order. Therefore, DSC and XRD data from FIGS. 2 and 3 are consistent with either a solid solution having very low crystalline order or a glass having an amorphous morphology.

In some embodiments, compositions of the invention provide an exposure (AUC_{(tf)}) of at least about 10,000 hr·ng/mL when: a composition of the invention; a formulation comprising a composition of the invention; or dosage form comprising a composition of the invention is administered to a human patient in an amount comprising the equivalent of about 100 mg of posaconazole free base under fasted conditions. In some embodiments, it is preferred to provide a composition of the invention in a particulate form by milling the solid composition, preferably milling it to a particulate form having a bulk density of greater than about 0.6 g/mL, more preferably a particulate form having a bulk density of from about 0.6 g/mL to about 0.7 g/mL as determined by gravimetric measurement of a measured volume of the particulate material. In some embodiments upon oral administration of an amount of a composition of the invention containing the equivalent of about 200 mg of posaconazole free base to a human patient under Fasted Conditions the composition provides a Cₘₐₓ plasma level of at least about 670 ng/mL. In some embodiments, compositions of the invention administered to a patient under fasted conditions in an amount providing the equivalent of from about 80 mg of posaconazole free-base to about 500 mg of posaconazole free-base, preferably from about 160 to about 250 mg posaconazole free-base provides a Cₘₐₓ in a patient to whom it is administered of at least about 335 ng/mL.

In another aspect of the invention, therapeutic or prophylactic treatment of a fungal infection is provided to a patient by orally administering: a composition of the invention; a formulation comprising a composition of the invention; or a dosage form comprising a composition of the invention in an amount which provides a Cₘₐₓ in a patient to whom it is administered of at least about 335 ng/mL. In some embodiments, therapeutic or prophylactic treatment of a fungal infection is provided to a patient in need thereof by orally administering: a composition of the invention; a formulation comprising a composition of the invention; or a dosage form comprising a composition of the invention in an amount which provides the equivalent of from about 80 to about 500 mg of posaconazole free base, preferably from about 160 to about 250 mg posaconazole free-base, to the patient. In some embodiments daily oral administration of: a composition of the invention; a formulation comprising a composition of the invention; or a dosage form comprising a composition of the invention in an amount which provides the equivalent of from about 80 mg of posaconazole free base to about 500 mg of posaconazole free base, preferably from about 160 to about 250 mg posaconazole free-base, is provided as a single or divided dose and is repeated for a period of time to target a steady-state C_{avg} plasma level of at least about 319 ng/mL in at least about 75% of a patient population to whom it is administered, or to target a Cavg of at least about 228 ng/mL in at least about 90% of a patient population to whom it is administered.

In some embodiments it is preferred to provide a dosage form for administering to a patient wherein the amount of posaconazole contained therein comprises from about 80% to about 125% of the desired amount of posaconazole free-base equivalent in accordance with FDA guidelines regarding manufacturing a medicament.

As mentioned in the background, posaconazole bioavailability exhibits a strong food effect. The label for Noxafil®, a commercially available form of posaconazole comprising crystalline posaconazole present in a medium in which it is dispersible, (see for example, the entry for Noxafil® in the Physicians Desk Reference (PDR), which is incorporated in its entirety by reference as if fully set forth herein) suggests that in the absence of being able to administer posaconazole orally to a patient under Fed Conditions, consideration should be given to treatment using another method.

Neutropenic patients, for example, those with prolonged neutropenia from chemotherapy, are often disadvantaged in their ability to intake food or nutritional supplements. This disability makes effective oral administration of posaconazole problematic. The inventors have found that oral administration of a formulation comprising a composition of the invention surprisingly eliminates the food effect, that is, oral administration of a formulation comprising a composition of the invention provides substantially the same posaconazole exposure and less variability in bioavailability across a patient population regardless of whether the formulation is administered under Fed Conditions or Fasted Conditions. Moreover, when the results of oral administration of a formulation comprising a composition of the invention are compared to those obtained after administration of an equivalent amount of posaconazole in the form of the commercially available formulation (Noxafil®), under either fed conditions or fasted conditions, the composition of the invention yields surprisingly increased bioavailability, with lower variability in bioavailability across a population of subjects, and higher exposure levels (AUC) in healthy volunteers to whom it is administered. Moreover, it is believed that similar results are achieved in patients to whom formulation comprising a composition of the invention is administered.

Moreover, oral administration of dosage forms comprising compositions of the invention yield remarkable and unexpected increases in both plasma levels and show less variability in bioavailability across a patient population to whom it is adminstered when compared to oral administration of compositions containing the same amount of posaconazole and the same polymer but which comparative compositions were prepared by spray-drying techniques. The inventors have noted that the compositions of the present invention have solid density in excess of 1.2 g/mL. The inventors have also found that when milled, compositions of the invention yield a particulate that has in excess of 0.6 g/mL bulk density when a measured volume of the particulate material is measured gravimetrically. In comparison, the inventors have noted that spray-dried compositions having the same ratio of posaconazole and polymer, and which have been prepared using the same polymer as the compositions of the invention exhibit a bulk density of less than about 0.3 g/mL.

In clinical studies the inventors have surprisingly found also that oral administration of a composition of the invention under either fed conditions or fasted conditions provides PK results substantially the same as those achieved by intravenous (IV) administration of an aqueous posaconazole suspension. Table I, below, presents the results of studies which summarize all of the foregoing information.

As is known for administration of the commercially available posaconazole suspension, bioavailability of posaconazole is generally optimized by administration of Noxafil® under “fed conditions”, which is reflected in the studies presented in the table above. The data in Table I show also that a formulation comprising a composition of the invention yields remarkably and unexpectedly improved PK parameters when administered under either “fed conditions” or “fasted conditions” in comparison to results achieved by administration of the aqueous suspension commercially available for oral administration administered under “fed conditions”.

TABLE I
Comparison of PK Parameters Observed After Administering 100 mg Dose of
Posaconazole

| | Study | | | | | |
|---|---|---|---|---|---|---|
| | | | | | A | B |
| | A | B | A | A | Capsule | IV^{c} |
| | Oral Sus | Oral Sus. | Tablet I^{a} | Tablet II^{a} | (Granulate)^{b} | suspension. |
| Parameter | (Fasted) | (Fed) | (Fasted) | (Fasted) | (Fasted) | (Fed) |
| Cmax^{d} | 84.0 | 243 | 385 | 358 | 335 | 443 |
| (ng/mL) | | | | | | |
| AUC (tf)^{de} | 2970 | 8470 | 11400 | 11000 | 10700 | 10100 |
| (hr · ng/mL) | | | | | | |
| AUC (I)^{df} | 3420 | 8750 | 11700 | 11300 | 11000 | 11100 |
| (hr · ng/mL) | | | | | | |
| CL/F^{g} | 34.0 | 12.5 | 9.16 | 9.25 | 9.67 | 10.1 |
| (L/hr) | | | | | | |
| t^{1/2} (hr) | 29.2 | 25.1 | 26.1 | 25.0 | 25.1 | 26.4 |

^{a}Capsules and Tablets I and II were prepared from a particulate form of a composition of the invention comprising hydroxypropylmethylcellulose acetate succinate (HPMC-AS, M grade) and posaconazole free base, which particulate was prepared by milling the extruded composition. Tablets were prepared by mixing the particulate form of the composition with ascorbic acid, PMCAS-M grade, silicon dioxide, magnesium stearate and either: (i) microcrystalline cellulose and low-substituted hydroxypropyl cellulose (Tablet I); or (ii) povidone and sodium croscarmellose (Tablet II), followed by tableting the mixture using direct compression.
^{b}Capsules were prepared by mixing the milled particulate form of the composition with ascorbic acid and HPMC-AS (M grade), and placing the mixture into gelatin capsules.
^{c}IV suspensions were prepared as described in Comparative Example 2 herein.
^{d}Values reported are average for the group studied.
^{e}AUC (tf), value end point determined at the point in time where sample contained minimum quantifiable amount of posaconazole, (LLOQ, lower limit of quantitation, 5.00 ng/mL using a liquid chromatography-tandem mass spectrometry method to quantify posaconazole present in plasma samples obtained from subject blood draws).
^{f}Values reported are calculated infinity values based on observed AUC (tf) values.
^{g}Apparent clearance calculated from AUC (I) values.

Further, these data show that the food effect observed using other oral formulations (exposure differences observed between administration of the dosage form under “Fed Conditions” in comparison to administration under “Fasted Conditions”) is substantially eliminated when formulations comprising a composition of the invention are orally administered. Table I further illustrates that oral administration of a composition of the invention yields PK parameters which are substantially the same as those available from intravenous administration (IV) of a posaconazole suspension to patients under “fed conditions”. Accordingly, in addition to the remarkable and unexpected improvements in exposure, a formulation for oral administration comprising a composition of the invention also eliminates the food effect observed with oral administration of the suspension. These impressive results are available from compositions of the invention whether the dosage form administered comprises the composition of the invention in the form of a milled particulate encapsulated in a gelatin capsule or is in the form of a tablet prepared by direct compression of an admixture of the milled particulate and various tableting excipients (see Table I results for tablets I and II in comparison with capsules filled with a composition of the invention).

Presented in Table II is the variation from the mean Cₘₐₓ and AUC values reported in Table I in the measured values observed across the subject population studied to obtain the data shown in Table I. The range of variation is expressed as a percentage of the ratio of one standard deviation of data to the mean value reported.

The data reported in Table II show that among fasted subjects, the oral suspension showed a broad range of variation between individual subjects. However, fasted subjects receiving a dosage comprising a composition of the invention unexpectedly have a significantly lower percentage of variability among the subjects studied.

TABLE II
Comparison of percentage variation from mean value in Parameters
Observed After Administering 100 mg Dose of Posaconazole

| | Study | | | | | |
|---|---|---|---|---|---|---|
| | | | | | A | B |
| | A | B | A | A | Capsule | IV Admin |
| | Oral Sus | Oral Sus. | Tablet I | Tablet II | (Granulate) | (Suspension) |
| Parameter | (Fasted) | (Fed) | (Fasted) | (Fasted) | (Fasted) | (Fed) |
| Cmax | 62% | 18% | 28% | 23% | 27% | 15% |
| (ng/mL) | | | | | | |
| AUC (tf) | 50% | 25% | 26% | 22% | 26% | 40% |
| (hr · ng/mL) | | | | | | |

The variability of measured PK parameters among subjects to whom Noxafil® (oral suspension of posaconazole) is administered is further illustrated by the data in Table III, which reports the steady-state PK results obtained by administration of 200 mg of Noxafil® aqueous suspension thrice/day (TID) to 194 patients afflicted with acute myelogenous leukemia (AML). Table III indicates that 99% of the group studied had a C_{avg} value of less than 1920 ng·hr/mL, 50% had a C_{avg} value of less than 486 ng·hr/mL and 5% had a C_{avg} value less than 170 ng·hr/mL. The mean C_{avg} in this patient population after administration of 200 mg Noxafil®/TID) was 582 ng/mL with a percentage variation of 64%. Due to the broad variability in bioavailability 200 mg TID was deemed a safe and effective dose, providing a therapeutic level for 90% of the patient population (C_{avg} value at or exceeding 228 ng/mL). This study indicates that variability in bioavailability across a patient population to whom Noxafil® was administered is at least as great as that observed in healthy human volunteers. It is believed that the unexpected significant reduction in variability in healthy subjects to whom a dosage comprising a composition of the invention was administered will be reflected in a significant reduction in variability among a patient population to whom a dosage comprising a composition of the invention is administered.

TABLE III

| Percentile | C_{avg} (ng · hr/mL) |
|---|---|
| Max | 1945 |
| 99% | 1920 |
| 95% | 1344 |
| 90% | 1080 |
| 75% | 719 |
| 50% | 486 |
| 25% | 319 |
| 10% | 228 |
| 5% | 170 |
| 1% | 103 |
| Min | 92 |

Based on the surprising reduction in variability and elimination of a food effect observed in administering a dosage comprising a composition of the invention, it is believed that when a composition of the invention is administered orally to patients needing posaconazole therapy in an amount of from about 80 mg posaconazole to about 500 mg posaconazole, preferably form about 100 mg posaconazole to about 400 mg posaconazole, per day in either a single or divided dose, there will be provided a safe and effective therapeutic plasma level of posaconazole. In some embodiments it is preferred to provide an amount of a composition of the invention and at an interval which will provide a steady-state C_{avg} at least about 319 ng/ml in 75% of the patients to whom it is administered. In some embodiments it is preferred to provide an amount of a composition of the invention and at an interval which will provide a steady-state C_{avg} of at least about 228 ng/ml in 90% of the patients to whom it is administered. In one embodiment it is particularly preferred to administer daily, in a single or divided dose a composition of the present invention to a patient in need thereof in an amount to provide from at least about 160 mg of posaconazole to at least about 250 mg of posaconazole, more preferably about 200 mg of posaconazole.

Table IV, below, shows PK results obtained by administering the indicated dose (in mg of posaconazole free base equivalent/Kg of subject weight) to cynomolgus monkeys under fasted conditions. Table IV compares the PK results observed after administration of various formulations with the PK results observed after administration of the commercially available Noxafil® posaconazole formulation.

Table IV shows that on a weight adjusted basis when the data presented therein are corrected for the differences in the amount of posaconazole administered to monkeys used in the study, and in comparison to material prepared by a spray-drying technique, unexpectedly both capsules and tablets comprising a composition of the invention provided significantly higher Cₘₐₓ levels and exposure (AUC) than is observed after oral administration of an equivalent amount of posaconazole contained in the other dosage forms used in the study.

TABLE IV

| | Formulation Administered | | | | |
|---|---|---|---|---|---|
| | | Capsule^{(d)} | Dosage Forms Containing | | |
| | Commercial | (granules from | HPMC-AS/Posaconazole | | |
| | Suspension | spray-dried | Composition of the Invention | | |
| | (Noxafil ®) | dispersion) | Capsule^{a} | Tablet I^{b} | Tablet II^{c} |
| PK Parameter | (13.2 mg/Kg | (16.1 mg/Kg | (12.7 mg/Kg | (9.8 mg/Kg | (12.7 mg/Kg |
| Studied | administered) | administered) | administered) | administered) | administered) |
| Tₘₐₓ | 4 | 4 | 26.8 | 7 | 4 |
| Cₘₐₓ | 19.8 | 91.9 | 137 | 172 | 202 |
| ng/mL/kg | | | | | |
| (dose adjusted) | | | | | |
| Cₘₐₓ/ | 1 | 4.6 | 6.9 | 8.7 | 10.2 |
| C_{max - susp.} | | | | | |
| (dose adjusted) | | | | | |
| AUC_{tf} | 373 | 2390 | 5200 | 6930 | 6240 |
| ng · hr/mL/mg | | | | | |
| (dose adjusted) | | | | | |
| AUCₘₐₓ/ | 1 | 6.4 | 13.9 | 18.6 | 16.7 |
| AUC_{max - susp.} | | | | | |
| (dose adjusted) | | | | | |
| t_{1/2} (hr) | 13.7 | 11.5 | 27.4^{e} | 22.5 | 23.1 |

^{a}Particulate prepared by milling an extrudate produced using a hot-melt extrusion technique, capsule prepared by sieving particulate and filing capsule with classified material providing a particle size range of from about 75 micron to about 300 micron with a median particle size of 250 microns.
^{b}Tablet I prepared by mixing the particulate form of the HPMC-AS/Posaconazole composition used in capsule preparation with ascorbic acid, HPMCAS-M grade, silicon dioxide, magnesium stearate, microcrystalline cellulose and low-substituted hydroxypropyl cellulose, followed by tableting the mixture using direct compression.
^{c}Tablet II prepared by mixing the particulate form of the HPMC-AS/Posaconazole composition used in capsule preparation with ascorbic acid, HPMCAS-M grade, silicon dioxide, magnesium stearate, povidone and sodium croscarmellose, followed by tableting the mixture using direct compression.
^{(d)}Capsules prepared by spray-drying a solution comprising posaconazole free base and a polymer as described in Comparative Example 1 herein.
^{e}data from half of study group excluded in calculating t_{1/2} due to late absorption evidenced by excluded subjects.

Table IV includes data obtained after administering to a monkey a dosage comprising a spray-dried composition prepared in accordance with the comparative Example 1 described herein. As shown in Table IV and in general, compositions provided by spray-drying do not provide high exposure levels, for example, as shown in Table IV, even though the compositions of the invention are administered at lower weight-adjusted dosage levels (10 mg/kg to 13 mg/kg for the present invention compositions of 16 mg/kg for spray-dried compositions), the AUC(tf) values observed are significantly higher than is observed when administering a spray-dried composition.

In Table IV, AUC_{tf} represents AUC over an interval from administration to the time of final quantifiable sample (the time at which a sample contains the minimum quantifiable level of posaconazole), and AUC(I) is a calculated projection of exposure at infinity based upon the value obtained from observed AUC_{tf}. The data in Table IV shows that oral administration under fasted conditions of dosage forms comprising a composition of the invention shows unexpected increases in Cₘₐₓ and exposure when compared with administration under fasted conditions of an equivalent amount of posaconazole contained in capsules filled with a composition prepared by a spray-dry technique.

Taken together, the data in Tables I to IV illustrate that oral administration of formulations comprising a composition of the present invention provide unexpected improvements in posaconazole plasma levels and posaconazole exposure in comparison to other dosage forms whether administered to subjects under fasted conditions of fed conditions, with less variability in observed PK values among a patient population to whom it is administered. Moreover, these data illustrate that the food effect seen with other orally administered posaconazole-containing formulations is substantially eliminated using formulations comprising a composition of the present invention.

As mentioned above, and without wanting to be bound by theory, it is believed that in compositions of the invention the posaconazole active pharmaceutical ingredient (API, in these formulations, posaconazole free-base) is dissolved in or molecularly dispersed in a polymer matrix. These compositions are believed to have a glass or solid solution morphology. Suitable polymers, or polymer mixtures, for use in the present invention are those that act as a solvent for posaconazole. One example of a class of suitable polymers is hydroxypropylmethylcellulose-derivative polymers. Moreover, suitable polymers for use in compositions of the invention yield a composition with posaconazole that has a glass transition temperature or melting point which is lower than the melting point of the posaconazole API itself and is capable of dissolving in vivo in the environment present within human intestines. In some embodiments it is preferred to employ a polymer or polymers in a composition of the invention that form a posaconazole API/polymer composition which has a melting point that is below the point of thermal decomposition of posaconazole. In some embodiments it is preferred to select a polymer or mixture of polymers for the composition that exhibit poor solubility in an aqueous environment having a pH-value which is more acidic than a value of about pH 2.0, and exhibits good solubility in an aqueous environment which is less acid than a pH value of from about 6.4 to about 6.8, preferably about pH 6.8.

Polymers meeting this pH-sensitive dissolution parameter which are suitable for use in a composition of the invention include, but are not limited to, hydroxypropylmethylcellulose-derivative polymers (HPMC-derivative polymers). Hydroxypropylmethylcellulose (HPMC) polymers, illustrated below as a polymer of Formula I, are cellulose polymers wherein “n” is an integer greater than 1, and “R” is independently for each occurrence to be hydrogen, —CH₃ or —CH₂—CH(OH)—CH₃, and wherein each “R” moiety occurs at least once within a given polymer strand.

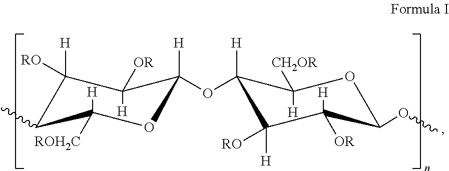

Accordingly, an HPMC-derivative polymer is an HPMC polymer wherein at least one or more of the “R” groups in a polymer strand is a hydrocarbon moiety other than methyl or hydroxypropyl, for example, phthalate, acetate, and succinate. Moreover, an HPMC-derivative polymer can include in addition, substitution at the hydroxyl group of a hydroxylpropyl moiety, for example, by esterification of the hydroxyl group with a substituent derived from an organic acid, for example, phthalate, acetate or succinate substituent. Examples of HPMC-derivative polymers suitable for use in preparing a composition of the invention include, but are not limited to, hydroxypropyl acetate succinate (HPMC-AS) polymer. An HPMC-AS polymer has the structure of Formula I wherein “R” is independently for each occurrence, H, —CH₃, —CH₂—CH(OH)—CH₃ (2-hydroxypropyl), —C(O)—CH₃ (acetate), —C(O)—(CH₂)₂—C(O)—OH (succinate), —CH₂—CH(CH₃)—OC(O)CH₃ (2-acetoxypropyl, derived from a 2-hydroxypropyl substituent having the 2-hydroxyl moiety substituted with acetate), or —CH₂—CH(CH₃)—OC(O)—(CH₂)₂—C(O)—OH (2-succinyl-propyl, derived from a 2-hydroxypropyl substituent having the 2-hydroxyl moiety substituted with succinate).

The inventors have surprisingly found that in selecting some grades of HPMC-AS polymer for use in compositions of the invention, the compositions can be prepared with little or no decomposition of the posaconazole used in the composition. Accordingly, in some embodiments using HPMC-AS polymer in the composition, it is preferred to prepare the composition utilizing a grade of HPMC-AS polymer that has a glass transition temperature which is from about 80° C. to about 145° C., preferably from about 100° C. to about 145° C., and more preferably from about 120° C. to about 135° C. Suitable HPMC-AS polymers which meet this criteria include, but are not limited to, HPMC-AS polymers having a degree of polymerization (expressed as a number average) of about 70. Suitable polymers are commercially available, for example, commercially available AQOAT® (Shin Etsu, Japan) materials having a number average of about 70 as measured with SEC-MALLS (in accordance with the manufacturers specifications). It will be appreciated that some compounds having a higher or lower number average can also be employed.

In some embodiments using HPMC-AS polymer, it is preferred to employ an HPMC-AS polymer having the acetyl moiety present in the polymer in a weight percent of from about 8 wt. % to about 12 wt. %, and the succinoyl moiety present in the polymer in a weight percent of from about 6 wt. % to about 15 wt. %. Suitable HPMC-AS polymers for use in the present invention are available commercially, for example, but not limited to, HPMC-AS supplied by ShinEtsu under its AQOAT® line of HPMC-AS polymers, for example, the L, M, and H grades of AQOAT® HPMC-AS. It will be understood that other grades of HPMC-AS, including those having different degrees of polymerization and percentages of succinoyl- and acetyl-substitution may be employed either alternatively or additionally without departing from the scope of a composition of the present invention.

In some embodiments, preferably the amounts of posaconazole (expressed in terms of the weight of the free-base form) and polymer employed in the composition are selected to provide a composition comprising from about 5 wt % posaconazole free-base equivalent to about 50 wt % posaconazole free-base equivalent. In some embodiments it is preferred to prepare compositions wherein posaconazole free-base and the polymer used is an HPMC-AS polymer, wherein the composition comprises a weight ratio of posaconazole free-base to HPMC-AS polymer of from about 1:1 to about 1:4. In some embodiments it is preferred to use an amount of posaconazole free-base to HPMC-AS weight ratio yielding a composition which is about 1:2 by weight to about 1:3 by weight posaconazole free base:HPMC-AS, more preferably the composition is about 1:3 by weight posaconazole free-base: HPMC-AS polymer.

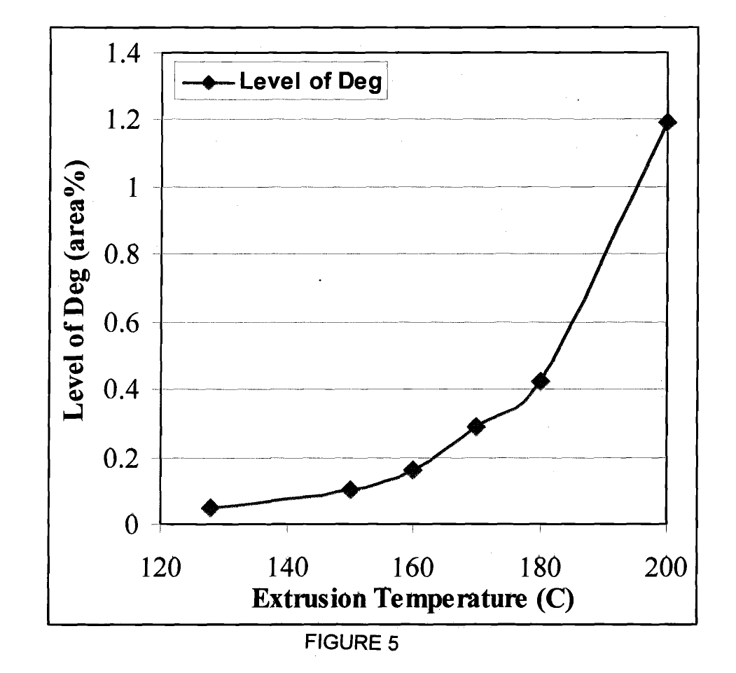

FIG. 5 illustrates the increasing amount of posaconazole degradation observed with increasing processing temperature when posaconazole free-base is dissolved in a molten polymer at a heating duration of from about 10 seconds to about 1.5 minutes. It can be seen from FIG. 5 that small increases in melt temperature increase the amount of degradation of the posaconazole free-base dramatically. Critically, this increase is greatest at temperatures greater than 10 degrees C. above the melting point of posaconazole free-base. The inventors have found surprisingly that admixtures comprising posaconazole free-base and an HPMC-derivative polymer behave as if they are a eutectic during heating. Without wanting to be bound by theory, it is believed that posaconazole free-base acts as a flux in admixture with an HPMC-derivative polymer, for example, HPMC-AS, to promote local melting of the polymer and dissolution of the posaconazole free-base into the polymer. Thus, surprisingly, compositions of the invention can be prepared by admixing a solid, particulate form of one or more polymers selected to comprise the polymer matrix of the composition with a solid, particulate form of posaconazole free-base, heating the admixture to its fluxing temperature or above so that a melt is formed in which the posaconazole free-base has dissolved, and cooling the melt to provide a solid. Preferably heating is limited to provide a temperature no greater than the fluxing temperature of the admixture and is maintained no longer than necessary to insure homogeneity of the composition before cooling the melt to provide a solid.

Accordingly, in some embodiments employing HPMC-AS as a matrix polymer, it is preferred to prepare a composition comprising posaconazole free base and an HPMC-AS polymer by a process comprising: (i) dry-blending a mixture of granules of posaconazole free base and granules of the selected hydroxypropylmethylcellulose acetate succinate polymer (HPMC-AS), wherein, preferably, the posaconazole is provided as a particulate material having a particle size of from about 1 micron to about 1 millimeter, and the polymer is provided in a powdered form having a particle size of from about 0.2 micron to about 1 micron, thereby forming an intimate mixture of polymer and posaconazole free base; (ii) heating the mixture to a temperature above the glass transition temperature (Tg) of the hydroxypropylmethylcellulose acetate succinate polymer employed and below the melting point of posaconazole free base (about 169° C.) and optionally blending the heated mixture, thereby forming a molten dispersion of the posaconazole free base dissolved in HPMC-AS; and (iii) cooling the dispersion formed in step (ii) to provide a composition of posaconazole free base in HPMC-AS. In some embodiments, optionally after Step (ii), the dispersion formed is extruded prior to carrying out cooling Step (iii). It will be appreciated that some forms of posaconazole other than the free base, for example, a posaconazole salt or prodrug, may be employed in this same process with similar results and not depart from the scope of the invention, provided that the form of posaconazole selected will perform a similar “fluxing” behavior when present in admixture with the polymer selected for use in preparing the composition of the invention. As mentioned herein, other polymers in which posaconazole is soluble and which having similar melting behavior may be used instead of or in addition to HPMC-AS polymers and still be within the scope of the present invention.

The inventors have surprisingly found that a composition produced according to the foregoing process minimizes or eliminates thermal decomposition and oxidation of the posaconazole free-base during the preparation of the posaconazole dispersion when compared to processes which utilize higher melting polymers, or mixtures of posaconazole and polymer in which posaconazole does not exhibit the fluxing properties described above, or when a process is utilized in which the polymer is melted and the other constituents are dissolved in the molten polymer. Accordingly, the inventors have surprisingly found that by using this process a composition of the invention can be prepared at a significantly lower temperature, and consequently using considerably less heat energy to prepare the composition, than would be employed by first melting a suitable polymer and then mixing the other constituents of the composition into the molten polymer constituent. Moreover, because posaconazole present in the admixture apparently acts as a fluxing agent promoting polymer melting, the time that the constituents of the composition must remain at temperature to provide a uniform composition can be minimized. The ability to minimize heat energy, temperature of the melt, and the amount of time the melt must be held at temperature to insure homogeneity translates to a surprising reduction in the amount of API that is degraded during formation of a composition of the invention in comparison to conventional hot-melt processes which rely on providing a molten polymer matrix into which the other constituents are dissolved.

In keeping with the foregoing discussion of preparatory processes, a melt can be prepared in any convenient apparatus in which an admixture of posaconazole and polymer can be heated and optionally stirred. Solidification can be carried out by merely cooling the melt by any means convenient and in any container convenient. Once a solid is obtained, the solid can be further mechanically processed to provide a convenient form for incorporation into a medicament, for example, tablets or capsules.

It will be appreciated that other methods of preparing a melt, solidifying it, and forming the solid into conveniently sized particles can be utilized without departing from the scope of the invention. For example, conveniently, compositions of the invention may be prepared using an extruder. When an extruder is employed to prepare compositions of the invention, conveniently, the material may be introduced into the extruder either in a pre-flux state, that is, as a dry admixture, or in a fluxed state, that is in a melted, plastic, or semi-solid state achieved after the application of sufficient heat to the admixture to cause the API to dissolve in the polymer, optionally when a fluxed charge is prepared, blending may be employed during heating to promote uniformity of the fluxed material.

If the material is introduced to the extruder in a fluxed state, residence time in the extruder is selected to be just sufficient to insure homogeneity of the composition and the temperature is preferably maintained in the extruder at a level just sufficient to insure that the material maintains its plasticity so that it can be extruded into a conveniently shaped extrudate. If the material is introduced into an extruder in a pre-flux state, the extruder components, for example, the barrels and any mixing chamber present in the equipment, will be maintained at a temperature sufficient to promote fluxing of the admixture. Temperatures selected for use in processing a composition will also take into account that blending which occurs within the extruder equipment, for example, in a mixing section of the barrels, will also contribute to localized fluxing of the admixture by imparting shear-stresses that induce heating in the mixture. Additionally it will be appreciated that equipment temperatures and residence times will be selected to minimize the amount of time that the admixture placed into the extruder spends under conditions of heating and/or shear stress so as to minimize the amount of API which is decomposed during formation of the composition, as discussed above. In general, extrusion processes in which heating is applied to the material extruded are termed “hot-melt/extrusion processes”.

When compositions of the present invention are prepared using extruder equipment, the extrudate thus provided can be in any convenient shape, for example, noodles, cylinders, bars, or the like. If desired, the extrudate can be further processed, for example by milling, to provide a particulate form of the composition.

The inventors have also surprisingly found that compositions prepared by melting an admixture of posaconazole and polymer produce a composition comprising posaconazole dissolved or molecularly dispersed in a polymer, and having a solid density of greater than about 1.2 g/mL. Even after milling to yield a particulate material having a particle size range of from about 75 microns to about 300 microns (which is equivalent to the size range of the granular material prepared by spray-drying technique described herein), the milled particles of the solid dispersion surprisingly have a bulk density of greater than about 0.6 g/mL, typically a bulk density of from about 0.6 g/mL to about 0.7 g/mL. when determined by weighing a measured volume of the particulate material produced by milling a sample of the composition of the invention. In contrast, spray-dried and milled particulate compositions (prepared by spray-drying a solution comprising the posaconazole and the same HPMC-derivative polymer used in providing a composition of the invention), when milled to the same particle size range typically have a bulk density of less than about 0.4 g/mL and typically less than about 0.3 g/mL when the bulk density is determined using the same technique.

The compositions of the invention can be administered to a patient either in the form it was produced, for example, a particulate, a prilled, or an extruded form, or the solid dispersion can be incorporated into a dosage form, for example, a tablet or capsule dosage form, by further processing. In some embodiments the composition in particulate form can be further admixed with one or more excipients, for example, extra-particle hydroxypropylmethylcellulose-derivative, for example, HPMC-AS (a binder which can also act as a diluent), povidone (binder), hydroxypropyl cellulose (binder), microcrystalline cellulose (diluent), low-substituted hydroxypropyl cellulose (disintegrant), sodium croscarmellose (disintegrant), silicon dioxide (glidant) and magnesium state (lubricant). After admixing with the desired excipient(s), the admixture can be compacted into a tablet using a standard tableting press. Alternatively, the milled composition can be used directly by filling it into a capsule, for example, a gelatin capsule. It will be appreciated also that a convenient dosage form may be prepared by directly filling a capsule with a melt comprising the composition of the invention, either in liquid or semi-solid form, and allowing the melt to solidify in the capsule. Using any one of these means, the present invention provides a dosage form comprising posaconazole for oral administration in a form about 3-fold to about 19-fold more bioavailable than is available from dosage forms comprising compositions prepared by spray-drying or other dosage forms, as illustrated above in Tables I through IV.

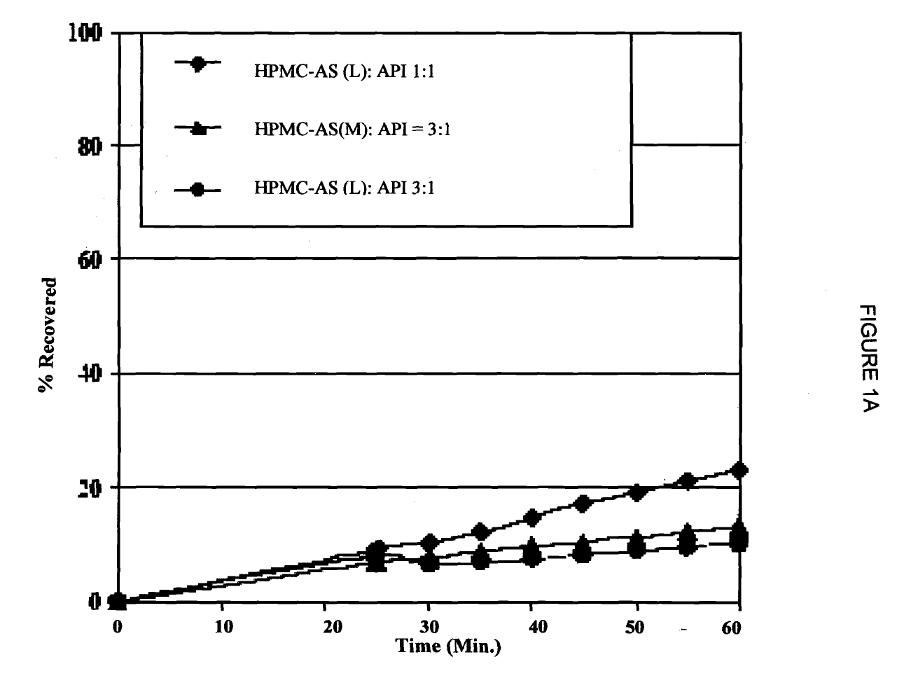

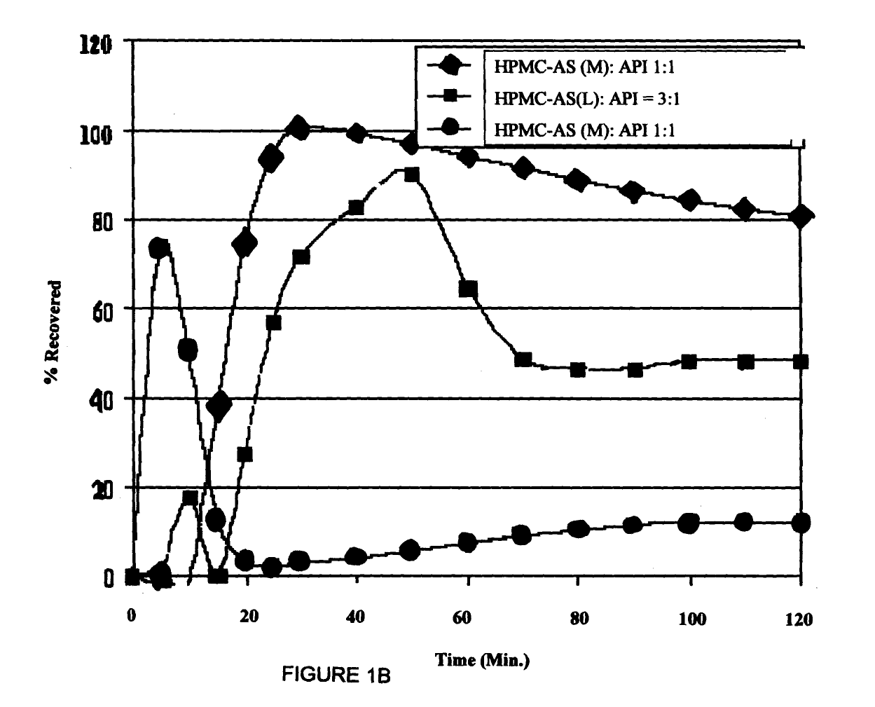

The inventors have surprisingly found that when compositions of the invention (posaconazole free base dissolved in or molecularly dispersed in an HPMC-derivative polymer, for example, an HPMC-AS polymer) were subjected to a dissolution tests using an aqueous dissolution media having a pH of about pH 1, the composition (and dosage forms comprising the composition) released less than about 20 w/w of the posaconazole present in the composition over a period of one hour, and when an aliquot of the same composition (or dosage form comprising the composition) was placed into a 50 mM aqueous phosphate buffer solution comprising sufficient amounts of NaH₂PO₄ and Na₂HPO₄ to provide a dissolution medium having a pH of from about pH 6.4 to about pH 6.8, the composition (or dosage form comprising the composition) released more than about 20 w/w of posaconazole within about 20 minutes of residing the less acidic dissolution medium. The inventors have found that similar dissolution results were obtained in a second determination carried out in a U.S.P. Paddle Dissolution Apparatus II in which the dissolution medium at the beginning of the test was a 0.1 N aqueous HCl solution. In this latter test, an aliquot of a composition of the invention (or a dosage form comprising a composition of the invention) was placed into the dissolution medium and stirred for about 1 hour while withdrawing aliquots of the dissolution medium and assaying them for posaconazole content. After one hour the acidity of the dissolution medium was adjusted to a pH of from about pH 6.4 to about pH 6.8 by the addition of a suitable quantity of a mixture NaH₂PO₄ and Na₂HPO₄ thus providing a dissolution medium comprising a 50 mM phosphate buffer solution in the stated pH range. Stirring was continued along with continued regular sampling and assaying of aliquots of the dissolution medium for posaconazole content. This latter test showed the same result, that in the more acidic medium, less than about 20 w/w of the posaconazole contained in the sample was released in one hour and after the pH of the dissolution medium was altered, more than about 20 w/w of the posaconazole contained in the sample was released within about 20 minutes of being placed in the less acidic environment. In either method of conducting these dissolution tests, determinations were carried out using a paddle speed of 50 rpm or 100 rpm and the dissolution solvent was maintained at 37° C. The inventors have also surprisingly found that these dissolution characteristics were maintained with different grades of HPMC-AS polymer, and in compositions employing the same grade of HPMC-AS polymer and different ratios of polymer and posaconazole. With reference to FIG. 1A, which shows the dissolution profile in a pH 1 environment of compositions comprising a 1:1 ratio of HPMC-AS MF-grade (diamond trace) 3:1 ratio of HPMC-AS MF-grade:posaconazole (triangle trace) and 3:1 ratio of HPMC-AS LF grade:posaconazole (solid circle trace), it can be see that only small quantities of the posaconazole contained in each composition were dissolved under the testing conditions described above. With reference to FIG. 1B, which shows the dissolution profile in a pH 6.4 environment (phosphate buffer) of compositions comprising a 1:1 ratio of HPMC-AS MF-grade (solid circle trace) a 3:1 ratio of HPMC-AS MF-grade:posaconazole (diamond trace) and 3:1 ratio of HPMC-AS LF grade:posaconazole (square trace), it can be see that a substantial amount of the posaconazole contained in each composition was dissolved under the testing conditions described in FIG. 1B. Thus FIGS. 1A and 1B illustrate that the compositions of the invention prevent dissolution of posaconazole in an acidic environment, for example, that found in a human stomach, and promote dissolution of posaconazole in a less acidic environment, for example, that found in a human intestine.

There follows non-limiting examples illustrative of the present invention but not limiting the present invention. In the examples below, the preparation of particulate materials from bulk solid compositions of the invention has been exemplified using a hammer mill, however, it will be appreciated that solid dispersions of the invention can be converted to a granular particulate form using any means, for example, by milling, prilling, or otherwise mechanically processing the solid dispersion to yield a particulate form.

### EXAMPLES

There follows examples of preparing a composition of the invention comprising posaconazole dispersed in HPMC-AS polymer, converting the solid composition of the invention into a pharmaceutical formulation and various dosage forms, and PK results obtained from administration of a formulation to human subjects.

### Example 1

### Preparation of an Extruded Composition of the Invention

### Example 1A

### Small Pilot-Plant Scale Extrusion Preparation

An admixture of posaconazole freebase and HPMC-AS polymer was prepared by blending together in a Bohle bin low shear blender 7.5 kg of HPMC-AS (M grade, Shin-Etsu AQOAT, as received from manufacturer having a particle size range of from about 5 microns to 1 millimeter) and an amount of material containing posaconazole free base assayed as equivalent of 2.5 kg of posaconazole free base (assay 25% active, total weigh 10.0 kg of material, micronized as received from the manufacturer, Schering-Plough corporation). The charge was blended until a homogeneous admixture was prepared.

Aliquots of the admixture prepared above were passed through a Leistritz ZSE twin screw extruder having 18 mm diameter, 450 mm long co-rotating screws until 10 Kg of extrudate comprising a composition of the invention had been prepared. During preparation of an extrudate, the admixture was fed into the extruder by a KCL-KT20 gravimetric feeder equipped with a 1:1 reducer and a 2-blade agitator. The outlet of the extruder was equipped with a die plate producing a 4 mm diameter “noodle” which was chopped at the outlet into random length pellets having a length of between 1 mm and 4 mm. In separate runs the die plate was selected from a die plate having a single 4 mm round opening or a die plate having double 4-mm round openings. The throughput rate was not affected by the selection of die plate. During extrusion the feeder agitator was operated at sufficient speed to provide an extrusion rate of 1.4 to 4.0 kg/h of the composition at the extruder outlet. The extruder screws were operated at 140 RPM during the extrusion process. At this speed, depending upon the feed rate of material into the extruder, the admixture and composition formed therefrom experiences a residence time of no more than 45 seconds, typically from 15 to 45 seconds, in the extruder. Accordingly, the admixture and melt formed therefrom experienced elevated temperatures in the extruder for a period of less than one minute during the extrusion process.

During the extrusion process, heat energy was supplied to the admixture while passing through the extruder from heating blocks secured along the barrel of the extruder. Power to the heating blocks was set to maintain a temperature of the extruder barrel between 120° C. and 135° C., as measured by thermocouples mounted inside of the extruder barrel. After the extrudate emerged from the extruder it was delivered via conveyor belt to a pelletizer, chopped, and the resultant pellets were left to further cool to the ambient temperature of the room air. During transportation on the conveyer belt the extrudate was fan cooled.

The cooled pellets from the previous step were milled in a Fitzmill hammer mill equipped with two different screen sizes: 0.065″ in a first milling step; and 0.020″ in a second milling step. The milled particles were classified through separate 50 mesh and 200 mesh screens in a mechanical screen sieves to isolate about 4.0 kg of particulate material having particle size in the range of from about 75 micron to about 300 micron. Particles in excess of 300 microns were recycled into the milling process. The particle fraction between 75 micron and 300 micron selected was used subsequently in the preparation of capsule and tablet dosage forms.

### Example 1B

### Large Pilot-Plant Scale Extruder Preparation

An admixture of posaconazole freebase and HPMC-AS polymer was prepared by charging a drum blender with 15.0 kg of HPMC-AS (M grade, Shin-Etsu AQOAT, granular, used as received) and an amount of material containing posaconazole free base assayed as equivalent of 5.0 kg of posaconazole free base (assay 25% active, total weigh 20.0 kg of micronized material used as received from the manufacturer). The charge was blended until a homogeneous admixture was prepared.

An extrudate was prepared from the admixture using a Berstorff twin screw extruder having 25 mm diameter, 700 mm long co-rotating screws. The extruder was fed by a KCL-KT40 gravimetric feeder equipped with a 1:1 reducer and a 2-blade agitator. The feeder was operated at sufficient speed to maintain an extrusion rate of 6.0 to 10.0 kg/h at the extruder outlet. The extruder screws were operated at 140 RPM to give the extruded material a residence time of 15 to 55 seconds in the extruder, consequently, the admixture was maintained at elevated temperature for less than one minute. The extruder was equipped with heating blocks along the barrel which were set to maintain a temperature of from 120° C. to 135° C. as measured by themocouples mounted within the extruder. The admixture previously prepared was placed into the hopper until a total of 20.0 Kg of admixture had been processed through the extruder

The outlet of the extruder was equipped with a die plate having double 4-mm round openings, forming the extrudate into twin 4 mm diameter “noodles” which were chopped at the outlet into random length pellets having a length of between 1 mm and 4 mm. The pellets were left to cool in the room air.

The dried pellets from the previous step were milled in a Fitzmill hammer mill using a 0.065″ screen in a first milling step and a 0.020″ screen in a second milling step. The milled product was collected and classified through separate 50 mesh and 200 mesh screens in a mechanical screen sieving operation. A 20.0 Kg cut of particulate material was thereby isolated having a particle size range of from about 75 micron to about 300 micron. Particles obtained in excess of 300 microns were recycled into the milling process. The particle fraction between 75 micron and 300 micron was used subsequently in the preparation of capsule and tablet dosage forms.

### Example 2

### Preparation of Tablets Comprising the Composition of the Invention

### Preparation of Tablest Designated “Tablet I”

Into a blender Bohle bin blender was placed 4 kg of the posaconazole-containing particulate material prepared in the previous example, 0.385 Kg of HPMC-AS (M grade, Shin-Etsu AQOAT, micronized, used as received) 0.5 kg of microcrystalline cellulose (Avicel PH102, NF grade, used as received), 0.4 kg of low-substituted hydroxypropyl cellulose (LH-B1, Shin-Etsu, used as received), and the charge was blended until a homogeneous powder admixture was obtained. Into the admixture was charged 0.11 kg of silicon dioxide, and the blending step was repeated. After a homogeneous powder admixture was again obtained, into the mixture was charged 0.025 kg of Magnesium stearate, and the admixture was blended again until homogeneous.

Aliquots of the blended homogeneous admixture prepared in the previous step weighing 550 mg were placed into a Hata-18 tableting press equipped with oval-shaped or capsule-shaped tablet die and pressed by direct compression into a tablet designated as type “Table I”.

### Preparation of Tablets Designated “Tablet II”

Into a blender Bohle bin blender was charged 4 kg of the posaconazole-containing particulate material prepared in the Example I, 0.385 kg of HPMC-AS (M grade, Shin-Etsu AQOAT, micronized, used as received) 0.4 kg of povidone, USP (USP Technologies, USP grade, used as received), 0.5 kg of sodium croscarmellose (FMC, NF grade, used as received), and the charge was blended until a homogeneous powder admixture was obtained. Into the admixture was charged 0.11 kg of silicon dioxide, and the blending step was repeated until the admixture was again homogeneous. Into the homogeneous admixture from the previous blending operation was charged 0.025 kg of Magnesium stearate, and the admixture was blended again until homogeneous.

Aliquots of the homogeneous admixture prepared in the last blending operation weighing 550 mg were placed into a Hata-18 tableting press equipped with a round tablet die and pressed by direct compression into a tablet designated as type “Tablet II”.

### Example 3

### Preparation of Capsules Comprising the Composition of the Invention

Into size 00 hard gelatin capsules (Swedish, orange) was placed 408 mg of the posaconazole-containing particulate material prepared in Example I, which had a particle size ranging from 75 microns to 300 microns. Capsules thus prepared were administered to subjects from which the data presented in Tables I, II, and IV, discussed herein, was obtained.

### Comparative Example 1

### Spray-Dried Dispersion

A spray-dried composition was prepared by spray-drying a solution comprising acetone/ethanol (2:1 v/v ratio) as a solvent (500 mL), posaconazole (75 mg free base equivalent) and 225 mg HPMC-AS (the same polymer used in the test composition of the invention). This solution was processed in a Nitro spray drying apparatus using a temperature of 85° C. and an air-flow of 80 LPM. After solids were obtained, residual solvent was removed from the solid granules by evacuating the isolated granules using a house vacuum (25″ Hg) with heating to 55° C. overnight. Once residual solvent had been reduced to a satisfactory level in this manner the particulate material was classified by retaining the material passing through a Mesh-50 screen (300 micron) and discarding the fraction of that material passing through a Mesh-200 (75 micron) screen. Accordingly, the material retained had a particle size ranged from 75 micron to 300 micron, and was utilized in preparing capsules for use in obtaining PK data.

Capsules were prepared by filling 400 mg aliquots of the resulting dried composition into size 00 capsules. These capsules were used in the studies described in Tables I, II, and IV herein.

### Comparative Example 2

### IV Suspension

A composition suitable for IV administration was prepared in accordance with Example 7 of Published U.S. patent application, Publication No. 2006/0160823, published Jul. 20, 2006 (which portion is specifically incorporated by reference as if fully set forth herein), but the formulation prepared in accordance therewith utilized the components in the amounts shown below in Table V:

TABLE V

| Ingredient | w/w % |
|---|---|
| Posaconazole | 5.90 |
| Sodium Phosphate Monobasic | 0.041 |
| USP | |
| Monohydrate | |
| Sodium Phosphate Dibasic USP | 0.126 |
| Anhydrous | |
| 1-palmitoyl-2-oleoyl- | 4.91 |
| phosphatidylcholine (POPC) | |
| Water | 89.00 |

This composition was employed for IV administration in the study described in Table I herein.

### Example 4

### PK Studies Using Dosage Forms Prepared in Examples 1 to 3 and Comparative Examples

In 4-way crossover studies comprising 2 parts (Fed and Fasted), PK data was obtained after administering posaconazole to 16 healthy human volunteers. In the first part (fasting conditions) the volunteers were administered a 100 mg oral suspension (Noxafil®) after an overnight fast of 10 hours. Subjects continued to fast for 4 hours after dosing and then received scheduled, standardized meals (similar content and portions). After a washout period, the volunteers were randomized into two groups and administered a 100 mg dose comprising either Tablet I or Tablet II, prepared in Example 2, above. After a second washout period, the 16 human volunteers were administered a 100 mg dose comprising the capsule prepared in Example 4, above.

In the second part of the study (Fed conditions), subjects received the study drugs in the same sequence with a standardized high-fat breakfast, which was consumed over 20 minutes. Study drug was administered approximately 10 minutes after the meal began (after half of the meal was consumed) and the second half of the meal was consumed in the remaining 10 minutes. For both parts, blood samples for the determination of posaconazole plasma pharmacokinetic concentrations were collected on before dosing, and at 0.5, 1, 2, 3, 4, 5, 6, 8, 12, 24, 48, 72, 96, 120, 144, and 168 hours after dosing.

AUC_{tf} and Cₘₐₓ, and Tₘₐₓ were determined from plasma concentrations of posaconazole (AUC_{tf} is area under the plasma concentration-time curve from time 0 to the time of the final quantifiable sample (defined herein above); Cₘₐₓ—maximum observed plasma concentration; Tₘₐₓ—time to the maximum observed plasma concentration), AUC(I), CL/F, and T1/2 were calculated, (AUC(I) is AUC from time 0 extrapolated to infinity beyond observed AUC(tf), CL/F—apparent oral clearance; T1/2—terminal phase half-life).

The results from these two studies are shown in Table VI. The values reported for Cₘₐₓ and AUC_{tf+} are mean of all volunteers. The geometric mean ratio of the fed and fasted Cₘₐₓ values for the suspension is 2.89 (Fed/Fasted) and for Tablet A, Tablet B, and the Capsule containing a composition of the invention the ratio is 0.85, 0.97, and 0.99 respectively (Fed/Fasted). The geometric mean ratio of the fed and fasted AUC_{tf} values for the suspension is 2.85 (Fed/Fasted) and for Tablet A, Tablet B, and the Capsule containing a composition of the invention the ratio is 1.03, 1.1, and 1.13 respectively (Fed/Fasted).

TABLE VI
Comparison of PK Parameters Observed After Administering 100 mg Dose of
Posaconazole at fasted and fed conditions in Clinical Studies of 16 Volunteers.

| | Comparative | | | | | | | |
|---|---|---|---|---|---|---|---|---|
| | Example | | Tablet or capsule dosage form comprising composition of | | | | | |
| | Oral Susp. | | the invention (100 mg posaconazole content) | | | | | |
| | (100 mg | | | | | | Capsule | |
| Parameter | posaconazole) | | Tablet A | | Tablet B | | (particulate fill) | |
| measured | Fasted | (Fed) | Fasted | Fed | Fasted | Fed | Fasted | Fed |
| Cmax | 84 | 243 | 385 | 327 | 358 | 348 | 335 | 330 |
| (ng/mL | | | | | | | | |
| AUC (tf) | 2970 | 8570 | 11,400 | 11,700 | 11,000 | 12,100 | 10,700 | 12,000 |
| (hr · ng/mL) | | | | | | | | |
| AUC (I) | 3,400 | 8750 | 11,700 | 11,900 | 11,300 | 12,400 | 11,000 | 12,300 |
| (hr · ng/mL) | | | | | | | | |

These data indicate that compositions of the invention are not markedly affected by food. When the PK data observed after administration of a composition of the invention are compared with PK values observed after administration of the oral suspension, the food effect that is observed using the oral suspension is substantially eliminated when utilizing a dosage form comprising a composition of the invention. Moreover, comparing the results shown in Table VI, with the results presented in Table I, above, confirms that compositions of the invention provide an unexpected increase in exposure and less variation in bioavailability over that which is observed with other posaconazole formulations administered under fasting condition, including compositions comprising posaconazole and a polymer which have been prepared by spray-drying technique.

From these studies it is expected that a composition of the invention will be useful in providing a therapeutic level of posaconazole in a patient to whom it is administered, whether in a fed or fasted state, if the composition is administered orally in an amount sufficient to provide a steady-state C_{avg} plasma level of at least about 319 mg/mL in at least about 75% of a patient population or a steady-state C_{avg} plasma level of at least about 228 ng/mL in at least about 90% of a patient population. It is expected that oral administration of at least about 80 mg daily, in a single or divided dose, preferably from about 80 mg to about 500 mg daily in a single or divided dose, more preferably from about 100 mg to about 400 mg daily in a single or divided dose, over a period of at least about 5 days will provide the desired steady-state C_{avg} plasma level.

This application claims the priority of U.S. Provisional Application No. 61/166,487, Filed Apr. 3, 2009, which application is incorporated by reference as if fully set forth herein in its entirety.

Various changes or modifications in the embodiments described herein may occur to those skilled in the art. These changes can be made without departing from the scope or spirit of the invention

1. A composition comprising posaconazole dissolved or molecularly dispersed in a hydroxypropylmethyl-cellulose-derivative polymer, wherein, when an amount of the composition comprising at least about 100 mg of posaconazole is administered orally to a human under fasted conditions, at least one of the following PK median parameters are observed: Cₘₐₓ in at least about 300 ng/mL; or an AUC(tf) of at least about 10,000 hr·ng/mL.

2. The composition of claim 1 wherein said HPMC-derivative polymer is a hydroxypropylmethylcellulose-acetate succinate polymer.

3. The composition of claim 1 which, when milled into a particulate having a particle size range of from about 300 microns to about 70 microns has a bulk density of at least about 0.4 g/cm³ as measured by gravimetric determination of a measured volume.

4. The composition of claim 3 having a bulk density of from about 0.4 g/cm³ to about 0.7 g/cm³.

5. The composition of claim 1 having an solid density of greater than about 1.2 g/mL.

6. A composition of claim 1 which is prepared as an extruded material.

7. A pharmaceutical formulation comprising the composition of claim 1 having the immediate release dissolution profile shown in FIG. 1B when dissolved in a pH 6.8 buffered media at 37° C. in a USP Apparatus II with a 100 rpm paddle speed.

8. The dosage form of claim 7 further having a dissolution profile shown in FIG. 1A when dissolved in a pH 1.0 buffered media at 37° C. in a USP Apparatus II with a 100 rpm paddle speed

9. A process for preparing a composition comprising posaconazole and hydroxypropylmethylcellulose acetate succinate, the process comprising:
(a) dry-blending HPMC-AS and posaconazole in a ratio of from about 1:1 (HPMC-AS:posaconazole) to about 4:1 (HPMC-AS:posaconazole) to form a homogeneous admixture;
(b) heating the admixture prepared in step “a” to a temperature below the glass transition temperature of the HPMC-AS present in the mixture and above the fluxing temperature of the mixture, thereby forming a melt, optionally while blending the admixture; and
(c) cooling the melt formed in Step “b”, thereby providing a solid composition comprising posaconazole dissolved or molecularly dispersed in HPMC-AS.

10. The process of claim 9, wherein, between steps “b” and “c” the melt is extruded to provide an extrudate of a desired cross-sectional shape.

11. The process of either of claim 9 wherein the solid composition provided by the process has an solid density of greater than about 1.2 g/mL.

12. The process of claim 11 wherein said solid composition has a dissolution profile substantially similar to that shown in FIG. 1B when dissolved in a pH 6.8 buffered media at 37° C. in a USP Apparatus II with a 100 rpm paddle speed.

13. The process of claim 9 wherein the solid composition is milled to form a granular composition having a particle size of from about 70 micron to about 350 micron and said granular composition has a bulk density of greater than about 0.4 g/mL as determined by weighing a measured volume of the particulate material.

14. A method of treating a fungal infection in a patient in need thereof comprising administering to the patient, for at least about 5 days, a pharmaceutical formulation or dosage form comprising an amount of the composition of claim 1 sufficient to target a steady-state C_{avg} plasma level of at least about 319 ng/mL in at least about 75% of a patient population or a C_{avg} of at least about 228 ng/mL in at least about 90% of a patient population.

15. The method of claim 14 wherein the amount of the composition administered comprises from about 100 mg to about 400 mg of posaconazole and is administered in a single or divided daily dose.

16. The method of claim 14 wherein the composition administered contains from about 80 mg of posaconazole to about 500 mg of posaconazole.

17. The method of claim 15 wherein the patient is neutropenic.

18. A pharmaceutical dosage form comprising a composition, said composition comprising posaconazole dissolved in or molecularly dispersed in a hydroxypropylmethyl-cellulose-derivative polymer, wherein, when an amount of the composition containing at least about 100 mg of posaconazole is administered orally to a human under fasted conditions, at least one of the following PK median parameters are observed: Cₘₐₓ in excess of about 300 ng/mL; or an AUC(tf) of at least about 10,000 hr·ng/mL.

19. The dosage form of claim 18 wherein the hydroxypropylmethyl-cellulose-derivative polymer is hydroxypropylmethyl-cellulose-acetate succinate.

20. (canceled)

21. (canceled)

22. The dosage form according to claim 18, wherein said dosage form is either a tablet or a capsule.
